(19) United States
(12) Patent Application Publication
(10) Pub. No.: US 2026/0102417 A1
(43) Pub. Date: Apr. 16, 2026

(54) COMPOSITIONS FOR THE TREATMENT AND PREVENTION OF CANCER

(71) Applicant: Keyzell Holding S.L., Sevilla (ES)

(72) Inventors: NABIL HAJJI, Sevilla (ES); Francisco Rodríguez, Sevilla (ES)

(21) Appl. No.: 19/116,641

(22) PCT Filed: Sep. 25, 2023
(86) PCT No.: PCT/IB23/59435

§ 371 Date: Mar. 28, 2025

(30) Foreign Application Priority Data
Sep. 29, 2022 (AU) 2022902823
Jun. 7, 2023 (UA) 2023901809

(51) Int. Cl. A61K 31/675; A61K 31/438; A61K 31/439; A61K 31/496; A61K 31/5377; A61K 39/00; A61K 45/06; A61P 35/00; C07K 16/28

(52) U.S. Cl. CPC A61K 31/675; A61K 31/438; A61K 31/439; A61K 31/496; A61K 31/5377; A61K 45/06; A61P 35/00; C07K 16/2818; A61K 2039/505

(57) ABSTRACT

The present invention relates to methods of treating or preventing cancer. In particular, the invention is directed to the use of selective non-peptide antagonists of the NK1 receptor (NK1R) in the treatment or prevention of cancer.

### FIELD OF THE INVENTION

The present invention relates to methods of treating or preventing cancer. In particular, the invention is directed to the use of high doses of aprepitant and derivatives thereof in the treatment or prevention of cancer.

### BACKGROUND OF THE INVENTION

Any discussion of the prior art throughout the specification should in no way be considered as an admission that such prior art is widely known or forms part of common general knowledge in the field.

Survival rates from cancers are often very closely linked to the frequency of early diagnosis, the aggressiveness of the cancer, the availability of effective anti-cancer therapies that are targeted to the cancer, the overall health of the subject undergoing treatment, and/or whether there are options, surgical or otherwise, for the removal of tumours and cancerous cells.

All types of cancer are generally characterized by uncontrolled cell division through aberrant molecular signalling that allows cells to circumvent cell-cycle arrest/apoptosis. The persistence of the cellular proliferation is often attributed to, at least in part, the disabling of cellular mechanisms for programmed cell death.

However, common to many cancers is the fact that indiscriminate therapeutic agent targeting, and the toxicity of the agents, limits the efficacy of anti-cancer therapies in their treatment.

To circumvent the targeting issue, while also producing therapies that have broad applicability, it can be useful to target cellular processes that are significantly increased in the cell proliferation associated with cancer.

One class of molecules involved in evasion of cell death and tumour promotion are the protein kinases, many of which are prolific and promiscuous kinases with vast arrays of substrates. Unfortunately, the inhibition of a protein kinase involved in multiple pathways can often have severe side-effects, which can be a barrier to successful drug development and commercialization.

One strategy to expedite the drug development pathway for new therapies is to repurpose (or reposition) existing licensed drugs for new medical indications. Repurposing is effectively the reuse of drugs with specific indications for new applications, while taking advantage of existing knowledge and safety data. Recent examples of promising repurposing of drugs for the treatment and prevention of cancer include the use of anti-parasitic and anti-psychotic drugs. In the case of cancer, particularly attractive drugs for repositioning may be those that are used in conjunction with cancer therapies to treat auxiliary symptoms or side-affects indirectly associated with the cancer or the cancer therapies themselves. This would be indicative of a tolerance of the drug in cancer patients, whose overall health is compromised and therefore likely to be more sensitive to medications.

Of course, while repurposing may allow expedition of some aspects of drug development as far as safety data is concerned, it does not circumvent the need to demonstrate the repurposed drug is actually effective for the new application via in vivo studies and human trials, as in vitro data is often not representative of in vivo results. Similarly, in vivo trails in model organisms, especially small mammals such as mice and rats, often do not translate to efficacy in humans. This is particularly true for the research in relation to anti-cancer compounds that may target complex interactions between different cell types such as cancer cells and immune cells that may be extremely difficult to reproduce during animal investigations.

This is evident from numerous reviews and articles that show there is a low likelihood of animal model results translating to human trials and clinical success. For example, a systematic review of highly cited animal studies revealed that only 37% of the studies were replicated with subsequent human trials, and 18% of the studies lead to contradictory results in humans (Hackam and Redelmeier, 2006, JAMA).

Another review directed specifically to the field of oncology showed that the average rate of successful translation from animal models to clinical cancer trials was less than 8% as at 2013 (WY Mak et al, 2014, Am J Transl Res). A more recent study limited to drugs being trialed for lung cancer could not establish any linear trend between the results achieved in preclinical trials and the subsequent approval of a lung cancer therapeutic (Pan et al, 2020, Frontiers in Oncology).

The reasons for the lack of successful translations from animal studies are many and varied, but one stumbling block is an understanding of how active dosages can fluctuate between in vitro and animal models, and applying calculators to determine an appropriate human dosage regime after an animal trial. It is very difficult to establish an appropriate dosage when transitioning from in vitro studies, which are often performed using immortalised cells in liquid media, to animal trials, which can be conducted using any number of administration methods.

However, once an effective dosage and administration method is successfully developed for an animal model in a pre-clinical trial (often expressed as a mg of drug per kilogram of the subject (mg/kg), per a period of time, such as a day), the determined mg/kg dosage cannot simply be applied to a human. Indeed, those skilled in the art would appreciate that there are algorithms that are used to extrapolate the dose used in an animal model to a human equivalent dose (HED). A safety factor of at least 10 is generally applied to the HED to ensure that the first does in a human trial will not cause adverse effects. However, despite these calculators being endorsed by the FDA (USFDA. Guidance for Industry: Estimating the Maximum Safe Starting Dose in Adult Healthy Volunteer. Rockville, MD: US Food and Drug Administration; 2005), they are still an approximation based on the average body surface areas of the animal model as a comparator to humans.

As such, not only is it understood that most drugs that demonstrate a desired activity at the preclinical stage in vitro and in vivo trials will not demonstrate the same activity in clinical trials because cell lines and animal models cannot mimic the complexity of human illness, it is also understood that the dosages used in animal models cannot be applied to humans without at least working out the HED.

Aprepitant (5-[[(2R, 3S)-2-[(1R)-1-[3,5-bis(trifluoromethyl)phenyl]ethoxy]-3-(4-fluorophenyl)-4-morpholinyl]methyl]-1,2-dihydro3H-1,2,4-triazol-3-one) is the selective non-peptide antagonist of the NK1 receptor (NK1R). NK1R is a receptor for Substance P (a neuro peptide composed of a chain of 11 amino acid residues), and NK1R agonists have been previously developed for the treatment of various disorders of the central and peripheral nervous system. NK1R antagonists such as aprepitant, rolapitant, casopitant, fosaprepitant, netupitant, and maropitant are effective and approved for the treatment of nausea and vomiting. In particular, the drug aprepitant (sold under the trade mark EMEND® by Merck) is approved and regularly used for the prevention of vomiting during chemotherapy, indicating it is well tolerated by cancer patients. It is given for three days at a recommended dose of 125 mg once on day 1 (1 hour prior to commencement of chemotherapy), and 80 mg once daily on days 2 and 3.

It was first suggested in 2010 that aprepitant may be a promising new target in the treatment of cancer, though this assessment was made purely on the basis of in vitro studies using tumour cell lines (Munoz and Rosso, 2010, Invest New Drugs). Despite this, and the fact that aprepitant has since been tested against a wide range of human cell lines and xenograft murine models, there have not been oncological clinical trials with humans to verify any of the reported in vitro and animal model results (Majkowska-Pilip et al., 2019, Pharmaceutics). To this point, there is no clear data that has established aprepitant as effective in the prevention/treatment of cancer in humans, let alone an appropriate human-specific dosage amount, means of administration or dosage regime.

In 2014, a study was published that demonstrated that a prodrug of aprepitant, fosaprepitant, showed some therapeutic efficacy against immunocompromised nude mice xenografted with the osteosarcoma cell line, MG-63, when injected weekly at a dose of 80 mg/kg (Munoz et al, 2014, International Journal of Oncology).

In another example, WO2015097322 describes the use of aprepitant for at a dose of 10-80 mg/kg per day, for period up to 28 days, for the treatment of immunocompromised nude mice with a range of provoked cancers and tumours. While this publication claims that the results presented demonstrate that treatment with aprepitant, at doses between 10 and 80 mg per kilogram of weight per day, reduces the size of cancerous tumours in mammals, it is actually only demonstrating that this particularly dosage regime is somewhat effective at 10-80 mg/kg/day in mice. Indeed, if a dose conversion between animal models and humans was applied to WO2015097322, the HED (human equivalent dose) of 10-80 mk/kg/day would be 0.81 mg/kg/day-6.5 mg/kg/day* (*obtained by dividing the mouse dose by 12.3 in accordance with body surface area method endorsed by FDA; Nair and Jacob, 2016, Journal of Basic and Clinical Pharmacy; Reagan-Shaw et al, 2007, FASEB). As such, for an average human of 70 kg, this would equate to about 57 to 455 mg of aprepitant per day.

Moreover, WO2015097322 does not actually reduce the size of cancerous tumours in mammals, as the results only demonstrate that the aprepitant slows the growth of the tumours without any net reduction in the tumour sizes. This does not correlate well with the previously reported in vitro studies with aprepitant, that showed much higher levels of anti-cancer activity against immortalized, cancerous cell lines. Arguably, WO2015097322 mainly teaches the use of aprepitant as a chemosensitizer to be used at low doses (1 mg/kg/day-10 mg/kg/day in mice; equating to 81 ng/kg/day-0.81 mg/kg/day in humans) in conjunction with an active chemotherapeutic agent (cisplatin), as is evidenced by the dosage ratios used in the presented examples 7 to 12. Thus, in terms of the in vivo trials of aprepitant for the treatment of cancer, the results have not been as encouraging as the in vitro trials, which probably accounts for the fact the drug has not progressed to clinical trials in humans.

In view of the foregoing, a preferable outcome of the present disclosure is to determine the potential of NK1R antagonists (particularly aprepitant) for preventing/treating cancer in humans.

### SUMMARY OF THE INVENTION

Surprisingly, it has been found that NK1R antagonists have therapeutic efficacy against cancer in humans. In embodiments, the NK1R antagonist is aprepitant. Further, in embodiments, the therapeutic efficacy is achieved when a high dose of NK1R antagonist is administered, preferably for a sustained period of time. The dose NK1R antagonist in this context is well beyond what has previously been used for the approved treatment of vomiting and nausea.

In particular, the present inventors are the first to demonstrate in human in vivo studies that a NK1R antagonist has selective cytotoxicity and can be used for the treatment and/or prevention of cancer, as opposed to merely enhancing the effect of chemotherapeutic drugs (as shown in murine trials at low doses), or in treating immortalized cancer cell lines in vitro.

In one aspect, there is provided a method of treating or preventing cancer in a human subject, the method comprising the step of administering to said human subject a composition comprising a therapeutically effective amount of a NK1R antagonist, or a derivative or pharmaceutically acceptable salt thereof. Preferably, the NK1R antagonist is selected from aprepitant, rolapitant, casopitant, fosaprepitant, netupitant or maropitant, or a derivative or pharmaceutically acceptable salt thereof.

In embodiments, the NK1R antagonist or a derivative or pharmaceutically acceptable salt thereof is administered at a dose of about 100 mg to about 10000 mg per day, preferably about 500 mg to 5000 mg per day.

In embodiments, the cancer is solid tumour or a blood cancer including, but not limited to, sarcomas, carcinomas, lymphomas, leukemia, myelomas and circulating tumour cells (CTCs).

In embodiments, the method further comprises administering the NK1R antagonist, or a derivative or pharmaceutically acceptable salt thereof, in combination with at least one bioactive agent, preferably wherein the at least one bioactive agent is an anticancer drug, chemotherapeutic agent, curcuminoid, or a prodrug, or a combination thereof.

In another aspect, there is provided use of a NK1R antagonist, or a derivative or pharmaceutically acceptable salt thereof, in the manufacture of a medicament for treating or preventing cancer in a human subject. The NK1R antagonist is preferably selected from aprepitant, rolapitant, casopitant, fosaprepitant, netupitant or maropitant, preferably aprepitant, or a derivative or pharmaceutically acceptable salt thereof.

### BRIEF DESCRIPTION OF THE DRAWINGS

FIG. 1 shows the use of the prodrug of aprepitant, fosaprepitant, in the treatment of mouse lung cancer 3LL cells in vitro. Cell viability was measured after 24 hours (t24) or 48 hours (t48).

FIG. 2 shows the use of the prodrug of aprepitant, fosaprepitant, in the treatment of mouse mammary gland adenocarcinoma 4T1 cells in vitro. Cell viability was measured after 24 hours (t24) or 48 hours (t48).

FIG. 3 shows the use of the prodrug of aprepitant, fosaprepitant, in the treatment of mouse melanoma B16F10 cells in vitro. Cell viability was measured after 24 hours (t24) or 48 hours (t48).

FIG. 4 shows the use of the prodrug of aprepitant, fosaprepitant, in the treatment of mouse colon carcinoma CT26WT cells in vitro. Cell viability was measured after 24 hours (t24) or 48 hours (t48).

FIG. 5 shows the use of the prodrug of aprepitant, fosaprepitant, in the treatment of mouse prostate cancer tumour PTEN cells in vitro. Cell viability was measured after 24 hours (t24) or 48 hours (t48).

FIG. 6 shows the use of the prodrug of aprepitant, fosaprepitant, in the treatment of human urinary bladder carcinoma RT112 cells in vitro. Cell viability was measured after 24 hours (t24) or 48 hours (t48).

FIG. 7 shows the use of aprepitant in the treatment of mouse lung cancer 3LL cells in vitro. Cell viability was measured after 24 hours (t24) or 48 hours (t48).

FIG. 8 shows the use of a prepitantin the treatment of human breast adenocarcinoma MCF7 cells in vitro. Cell viability was measured after 24 hours (t24) or 48 hours (t48).

FIG. 9 compares the antitumor effectiveness of NK1R antagonist drugs aprepitant and netupitant for the treatment of mouse prostate cancer tumor in vitro. Cell viability was measured after 24 hours (t24) or 48 hours (t48).

FIG. 10 shows that aprepitant and doxorrubicine, in combination, act synergistically in the treatment of mouse prostate cancer tumor cells PTEN. Cell viability was measured after 24 hours (t24) or 48 hours (t48).

FIG. 11 shows that treatment with fosaprepitant-dimeglumine or Maropitant reduces tumoral growth in the mouse melanoma model.

FIG. 12 shows that combination of Fosaprepitant-dimeglumine and anti-PD1 therapies shows enhanced antitumoral activity compared with single treatments in mouse melanoma model.

### DETAILED DESCRIPTION

### Definitions

The terms “a,” “an,” “the” and similar referents used in the context of describing the invention (especially in the context of the following claims) are to be construed to cover both the singular and the plural, unless otherwise indicated herein or clearly contradicted by context.

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention otherwise claimed. No language in the specification should be construed as indicating any non-claimed element is essential to the practice of the invention, unless otherwise indicated herein or clearly contradicted by context.

Except where otherwise indicated, all numbers expressing quantities of ingredients, reaction conditions, and so forth used in the specification and claims are to be understood as being modified in all instances by the term ‘about’. Accordingly, unless indicated to the contrary, the numerical parameters set forth in the following specification and attached claims are approximations that may vary depending upon the desired properties sought to be obtained by the present invention. At the very least, each numerical parameter should be construed in light of the number of significant digits and ordinary rounding conventions. The term “about” may be understood to refer to a range of +/−10%, such as +/−5% or +/−1% or, +/−0.1%.

Recitation of ranges of values herein is merely intended to serve as a shorthand method of referring individually to each separate value falling within the range. Unless otherwise indicated herein, each individual value is incorporated into the specification as if it were individually recited herein. For example, if a range is from about 1 to about 50, it is deemed to include, for example, 1, 7, 34, 46.1, 23.7, or any other value or range within the range.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Unless the context clearly requires otherwise, throughout the description and the claims, the words “comprise”, “comprising” and the like are to be construed in an inclusive sense as opposed to an exclusive or exhaustive sense; that is to say, in the sense of “including, but not limited to”.

The term “treatment”, and the like, in the context of the present specification includes the alleviation of the symptoms associated with a cancer, cancer regression and/or remission. In certain embodiments a treatment will slow, delay or halt the proliferation of cancerous cells, or metastasis of a cancer, slow, delay or halt the increase in a tumour size that would ordinarily occur from the cellular proliferation within the tumour, prevent differentiation of a cell line, reduce tumour size, or reverse the progression of one or more tumours, at least temporarily. The treatment may cure the cancer, or delay morbidity. Hence, in the context of this invention the word “treatment” or derivations thereof when used in relation to a therapeutic application includes all aspects of a therapy, such as the alleviation of pain associated with the cancer being treated, alleviation of the severity of the cancer being treated, improvement in one or more symptoms of the cancer being treated, improvement in the overall well-being of the subject being treated. Use of the word “treatment” or derivatives thereof will be understood to mean that the subject being “treated” may experience any one or more of the aforementioned benefits. In general, the treatment may be related to the death of proliferating cells present in the cancer.

The term “prevention”, and the like, in the context of the present specification refers to the prevention of the recurrence of all or some of the symptoms associated with a cancer after a remission of said cancer, as well as the prevention of the formation of one or more cancers due to, for example, the metastasis of a cancer. The prevention may prevent morbidity due to one or more cancers, or delay morbidity due to one or more cancers. In general, the prevention may be related to the death of proliferating cells that may cause a cancer or cause a cancer to spread or recur.

In the context of this specification, where a range is stated for a parameter it will be understood that the parameter includes all values within the stated range, inclusive of the stated endpoints of the range.

In the context of the present invention, the term “subject” refers to a human, who has experienced and/or exhibited at least one symptom associated with a cancer. Typically, the subject is an individual having cancer and is under the clinical care of a medical practitioner. Further, as used herein, a “subject in need thereof” may additionally be a subject who has not exhibited any symptoms of a cancer, but who has been deemed by a physician, clinician or other medical profession to be at risk of developing cancer. For example, the subject may be deemed at risk of developing cancer (and therefore in need of prevention or preventive treatment) as a consequence of the subject's medical history, including, but not limited to, family history, pre-disposition, co-existing/contributory cancer.

In the context of the present invention, by “pharmaceutically acceptable excipient or diluent” is meant any excipient or diluent that is not biologically undesirable, i.e., the material may be incorporated into a pharmaceutical composition of the present invention and administered to a subject without causing any undesirable or undue biological effects, including but not limited to undesirable or undue toxicity, incompatibility, instability, irritation, allergic response and the like. In a preferred embodiment, the excipient or diluent is approved or approvable by a regulatory agency or body, (the regulatory agency or body being, for example, a Federal or State government), or listed in the U.S. Pharmacopoeia or other generally recognized pharmacopoeia for use in subjects.

Further examples of the invention are described below. However, it should be noted that the invention should not be limited to these examples, and that the invention is susceptible to variations, modifications and/or additions other than those specifically described, and it is to be understood that the invention includes all such variations, modifications and/or additions which fall within the scope of the claims.

NK1R Antagonists have Therapeutic Efficacy Against Cancer in Humans

In a first aspect of the present invention, there is provided a method of treating or preventing cancer in a human subject, the method comprising the step of administering to said human subject a composition comprising a therapeutically effective amount of a NK1R antagonist, or a derivative or pharmaceutically acceptable salt thereof. Preferably, the NK1R antagonist is aprepitant.

In a second aspect of the present invention, there is provided a method of inducing apoptosis in proliferating cancer cells in a tumour in a human subject, the method comprising the step of administering to said human a composition comprising a therapeutically effective amount of a NK1R antagonist, or a derivative or pharmaceutically acceptable salt thereof. Preferably, the NK1R antagonist is aprepitant.

In a third aspect of the present invention, there is provided a method of inducing cell death in cancer cells in a tumour in a human subject, the method comprising the step of administering to said human a composition comprising a therapeutically effective amount of a NK1R antagonist, or a derivative or pharmaceutically acceptable salt thereof. Preferably, the NK1R antagonist is aprepitant.

In a fourth aspect of the present invention, there is provided a method of inducing cell death in cancer cells for the treatment or prevention of cancer in a human subject, the method comprising the step of administering to said human a composition comprising a therapeutically effective amount of a NK1R antagonist, or a derivative or pharmaceutically acceptable salt thereof. Preferably, the NK1R antagonist is aprepitant.

In a fifth aspect of the present invention, there is provided a method of reducing the size of a tumour in a human subject, the method comprising the step of administering to said human a composition comprising a therapeutically effective amount of a NK1R antagonist, or a derivative or pharmaceutically acceptable salt thereof, wherein the administration reduces the tumour size by inducing apoptosis in proliferating cancer cells in the tumour. Preferably, the NK1R antagonist is aprepitant.

In a sixth aspect of the present invention, there is provided a method of treating or preventing cancer in a human subject, the method comprising the step of administering to said human 500 mg-5000 mg per day of a NK1R antagonist, or a derivative or pharmaceutically acceptable salt thereof. Preferably, the NK1R antagonist is aprepitant.

In a seventh aspect of the present invention, there is provided a method of inducing apoptosis in proliferating cancer cells in a tumour in a human subject, the method comprising the step of administering to said human 500 mg-5000 mg per day of a NK1R antagonist, or a derivative or pharmaceutically acceptable salt thereof. Preferably, the NK1R antagonist is aprepitant.

In an eight aspect of the present invention, there is provided a method of inducing cell death in cancer cells in a tumour in a human subject, the method comprising the step of administering to said human 500 mg-5000 mg per day of a NK1R antagonist, or a derivative or pharmaceutically acceptable salt thereof. Preferably, the NK1R antagonist is aprepitant.

In a ninth aspect of the present invention, there is provided a method of inducing cell death in cancer cells for the treatment or prevention of cancer in a human subject, the method comprising the step of administering to said human 500 mg-5000 mg per day of a NK1R antagonist, or a derivative or pharmaceutically acceptable salt thereof. Preferably, the NK1R antagonist is aprepitant.

In a tenth aspect of the present invention, there is provided a method of reducing the size of a tumour in a human subject, the method comprising the step of administering to said human 500 mg-5000 mg per day of a NK1R antagonist, or a derivative or pharmaceutically acceptable salt thereof, wherein the administration reduces the tumour size by inducing apoptosis in proliferating cancer cells in the tumour. Preferably, the NK1R antagonist is aprepitant.

Aprepitant has the chemical formula C₂₃H₂₁F₇N₄O₃, and the chemical name, 5-[[(2R, 3S)-2-[(1R)-1-[3,5-bis(trifluoromethyl)phenyl]ethoxy]-3-(4-fluorophenyl)-4-morpholinyl]methyl]-1,2-dihydro3H-1,2,4-triazol-3-one.

Aprepitant is also known as the trade name, EMEND®. EMEND® specifically refers to the prodrug of aprepitant, fosaprepitant dimenglumine, which has the chemical formula, C₂₃H₂₂F₇N₄O₆P·2(C₇H₁₇NO₅), and the chemical name 1-Deoxy-1-(methylamino)-D-glucitol[3-[[(2R,3S)-2-[(1R)-1-[3,5-bis(trifluoromethyl)phenyl]ethoxy]-3-(4-fluorophenyl)-4-morpholinyl]methyl]-2,5-dihydro-5-oxo-1H-1,2,4 triazol-1-yl]phosphonate (2:1) (salt). As used herein, it would be understood that the trade name “EMEND®” (or alternatively “IVEMEND” as branded in Europe) may refer to aprepitant, fosaprepitant dimenglumine or fosaprepitant, and these names may be used interchangeably unless indicated otherwise.

EMEND® is a common antiemetic agent that has been approved and used effectively for the treatment of nausea and vomiting during chemotherapy since its FDA approval in 2003. In recent times, it has been found to demonstrate antitumour activity in pre-clinical trials, though the in vitro and in vivo results for aprepitant have not correlated well, and it has never been the subject of a clinical trial in humans.

In the method of the invention, the cancer may be any cancer that presents as a solid tumour or a blood (liquid) cancer including, but not limited to, sarcomas, carcinomas, lymphomas, leukemia, myelomas and circulating tumour cells (CTCs). For example, the carcinoma may be that of the pancreas, bladder, breast, colon, mesothelioma, kidney, liver, lung, including small cell lung cancer, non-small cell lung cancer, head and neck, oesophagus, gall bladder, ovary, stomach, cervix, thyroid, prostate or skin. In general, the cancer will be characterized by uncontrolled cellular proliferation.

In other examples, the lymphoma may be B-cell lymphoma, T-cell lymphoma, Hodgkin's lymphoma, non-Hodgkin's lymphoma, hairy cell lymphoma, mantle cell lymphoma, myeloma, Burkett's lymphoma, or an extranodal lymphoma of the stomach, breast or brain.

The sarcoma may, for example, be fibrosarcoma, rhabdomyosarcoma, chondrosarcoma, leiomyosarcoma, mesothelial sarcoma, angiosarcoma, liposarcoma, bone tumours and tumours of the central and peripheral nervous system, including astrocytoma, neuroblastoma, glioma and schwannomas, or other tumours, including melanoma, seminoma, teratocarcinoma, osteosarcoma, xenoderoma pigmentosum, keratoctanthoma, thyroid follicular cancer and Kaposi's sarcoma.

The myeloma may be, for example, plasma cell myeloma or Kahler's disease or multiple myeloma. In other examples, the leukemia may be myelogenous leukemia, granulocytic leukemia, lymphatic leukemia, lymphocytic leukemia or lymphoblastic leukemia, polycythemia vera or erythremia.

In other non-limiting examples, the cancer may be, for example, acute lymphoblastic leukemia, acute myeloid leukemia, adrenocortical carcinoma, HIV and AIDS-related cancers, primary CNS lymphoma, anal cancer, gastrointestinal carcinoid tumours, brain astrocytomas, atypical teratoid/rhabdoid tumours, basal cell carcinoma, bile duct cancer, ewing sarcoma, osteosarcoma, malignant fibrous histiocytoma, brain glioma, bronchial tumours, cardiac tumours, embryonal tumours, germ cell tumours, cholangiocarcinoma, chordoma, chronic lymphocytic leukemia, chronic myelogenous leukemia, chronic myeloproliferative neoplasms, colorectal cancer, craniopharyngioma, cutaneous T-cell lymphoma, mycosis fungoides, Sézary syndrome, ductal carcinoma in situ (DCIS), uterine cancer, ependymoma, esthesioneuroblastoma, extragonadal germ cell tumours, eye cancer, intraocular melanoma, retinoblastoma, fallopian tube cancer, gastric cancer, gastrointestinal stromal tumours, testicular cancer, hypopharyngeal cancer, lip, mouth and oral cavity cancer, male breast cancer, merkel cell carcinoma, midline tract carcinoma with NUT gene changes, multiple endocrine neoplasia syndromes, myelodysplastic syndromes, nasal cavity and paranasal sinus cancer, nasopharyngeal cancer, oropharyngeal cancer, pancreatic neuroendocrine tumours, papillomatosis, paraganglioma, parathyroid cancer, penile cancer, pharyngeal cancer, pheochromocytoma, pituitary tumours, pleuropulmonary blastoma, primary peritoneal cancer, salivary gland cancer, vascular tumours, urethral cancer, uterine sarcoma, vaginal cancer, vulvar cancer, and Wilms tumour.

The methods of the present invention may prevent, delay or retard the development of cancers that, for example, may ordinarily develop from the metastasis of any of the cancers mentioned above. The methods may also prevent, delay or retard the recurrence of any of the cancers mentioned above after treatment.

The cancer may also be any cancer wherein the proliferative capacity of the cancer cells is modulated by a protein in any way. In some embodiments, the cancer may be any cancer wherein the cancer cells overexpress protein. It would be understood that by “overexpress” is meant that the cancer cell expresses more protein than a healthy cell of the same tissue or cell type. For example, a cancerous cell present in a breast tumour would be considered to overexpress protein if, when compared to a non-cancerous breast tissue cell, the expression level of protein in the cancerous cell present in a breast tumour was greater. Expression levels of protein in a cell may be determined by the skilled addressee using well known techniques, such as, but not limited to, mRNA quantitation, immunofluorescence and western blotting. In embodiments of the invention, the cancer cells, or cells in the cancerous microenvironment, overexpress the NK1 receptor.

While the composition of the invention may be used alone for effectively treating and preventing cancer, in some embodiments of the invention, the method comprises administering to a subject in need thereof a composition comprising a NK1R antagonist (or a derivative or salt thereof), and an additional bioactive agent. The bioactive agent may be included in a composition comprising the NK1R antagonist, or the bioactive agent may be co-administered with a composition comprising the NK1R antagonist. By co-administered is meant that the bioactive agent and composition may be administered at the same time, or the composition and the agent may be administered at, for example, alternating times, or in advance of each other, or in follow-up to each other, or combination thereof (i.e., pre-administration or post-administration is included in the meaning of the term co-administration). For example, the bioactive agent may be administered in advance of the composition, and also administered at the same time or for the same duration as the composition. By “same time” is not meant to be limited to an actual time, but rather a time-frame or duration. For example, a bioactive agent may be administered to a subject at the same time as a composition of the invention, whereby this means that the bioactive agent is administered according to any suitable schedule for a particular period (days, weeks, months or years), while the subject is also receiving a composition of the invention at the same or different schedule for the same particular period (i.e., the patient maybe be receiving a daily dose of the composition of the invention for three months, while, during that three months, the subject is receiving a weekly does of the bioactive agent).

The bioactive agent may be any compound having biological activity with respect to the cancer, including therapeutic activity. The bioactive agent may be capable of binding or interacting with the cells of the cancer. The bioactive agent may be any agent, drug, compound or composition that may be used for the detection, prevention and/or treatment of a cancer. In embodiments of the present invention, the bioactive agent is a therapeutic agent, and more preferably, a chemotherapeutic agent.

In particularly preferred embodiments of the invention, the bioactive agent is an anticancer drug or chemotherapeutic agent, or combination thereof. Further preferably, the bioactive agent is selected from the group consisting of Abiraterone acetate, Albumin-bound (nab) paclitaxel, Alemtuzumab, Altretamine, Asparaginase, Bendamustine, Bevacizumab, bleomycin, Bortezomib, Brentuximab vedotin, Busulfan, Cabazitaxel, Capecitabine, Carboplatin, Carmustine, Cetuximab, Chlorambucil, Cisplatin, Cladribine, Crizotinib, Cyclophosphamide, Cytarabine (Ara-C), Dacarbazine, Dactinomycin, Dasatinib, Daunorubicin, DaunoXome (liposomal daunorubicin), DepoCyt (liposomal cytarabine), Docetaxel, Doxil (liposomal doxorubicin), Doxorubicin, Eribulin mesylate, Erlotinib, Estramustine, Etoposide, Everolimus, Floxuridine, Fludarabine, Fluorouracil, Gefitinib, Gemcitabine, Gliadel wafers, Hydroxyurea, Ibritumomab, Ibritumomab, Idarubicin, Ifosfamide, imatinib, Ipilimumab, Irinotecan, Ixabepilone, Lapatinib, Lenallidomide, Lomustine, Mechlorethamine, Melphalan, Mercaptopurine, Methotrexate, Mitomycin, Mioxantrone, MG132, Nilotinib, Oxaliplatin, Paclitaxel, Panitumumab, Pazopanib, Peginterferon alfa-2b, Pemetrexed, Pentostatin, Pralatrexate, Procarbazine, Rituximab, Romidepsin, Ruxolitinib, Sipuleucel-T, Sorafenib, Streptozocin, Sunitinib, Temozolomide, Temsirolimus, Teniposide, Thalidomide, Thioguanine, Thiotepa, Topotecan, Tositumomab, Trastuzumab, Valrubicin, Vandetanib, Vemurafenib, Vinblastine, Vincristine and Vinorelbine.

Anticancer drugs may include 3F8, 8H9, Abagovomab, Abituzumab, Adecatumumab, Afutuzumab, Alacizumab pegol, Altumomab pentetate, Amatuximab, Anatumomab mafenatox, Anetumab ravtansine, Apolizumab, Arcitumomab, Ascrinvacumab, Atezolizumab, Bavituximab, Bectumomab, Belimumab, Bevacizumab, Bivatuzumab mertansine, Blinatumomab, Brentuximab vedotin, Cantuzumab mertansine, Cantuzumab ravtansine, Capromab pendetide, Carlumab, Catumaxomab, cBR96-doxorubicin immunoconjugate, Cetuximab, Citatuzumab bogatox, Cixutumumab, Clivatuzumab tetraxetan, Codrituzumab, Coltuximab ravtansine, Conatumumab, Dacetuzumab, Dalotuzumab, Daratumumab, Demcizumab, Denintuzumab mafodotin, Derlotuximab biotin, Detumomab, Drozitumab, Durvalumab, Dusigitumab, Ecromeximab, Edrecolomab, Elgemtumab, Elotuzumab, Emactuzumab, Emibetuzumab, Enavatuzumab, Enfortumab vedotin, Enoblituzumab, Ensituximab, Epratuzumab, Ertumaxomab, Etaracizumab, Farletuzumab, FBTA05, Ficlatuzumab, Figitumumab, Flanvotumab, Galiximab, Ganitumab, Gemtuzumab ozogamicin, Girentuximab, Glembatumumab vedotin, Ibritumomab tiuxetan, Icrucumab, Igovomab, IMAB362, Imalumab, Imgatuzumab, Indatuximab ravtansine, Indusatumab vedotin, Inotuzumab ozogamicin, Intetumumab, Ipilimumab, Iratumumab, Isatuximab, Labetuzumab, Lambrolizumab, Lexatumumab, Lifastuzumab vedotin, Lilotomab satetraxetan, Lintuzumab, Lorvotuzumab mertansine, Lucatumumab, Lumiliximab, Lumretuzumab, Mapatumumab, Margetuximab, Matuzumab, Milatuzumab, Minretumomab, Mirvetuximab soravtansine, Mogamulizumab, Moxetumomab pasudotox, Nacolomab tafenatox, Naptumomab estafenatox, Narnatumab, Necitumumab, Nesvacumab, Nimotuzumab, Nivolumab, Nofetumomab merpentan, Obinutuzumab, Ocaratuzumab, Ofatumumab, Olaratumab, Onartuzumab, Ontuxizumab, Oportuzumab monatox, Oregovomab, Otlertuzumab, Panitumumab, Pankomab, Parsatuzumab, Pasotuxizumab, Patritumab, Pembrolizumab, Pemtumomab, Pertuzumab, Pidilizumab, Pinatuzumab vedotin, Polatuzumab vedotin, Pritumumab, Racotumomab, Radretumab, Ramucirumab, Rilotumumab, Rituximab, Robatumumab, Sacituzumab govitecan, Samalizumab, Satumomab pendetide, Seribantumab, SGN-CD19A, SGN-CD33A, Sibrotuzumab, Siltuximab, Sofituzumab vedotin, Tabalumab, Tacatuzumab tetraxetan, Taplitumomab paptox, Tarextumab, Tenatumomab, Teprotumumab, Tetulomab, TGN1412, Ticilimumab (=tremelimumab), Tigatuzumab, TNX-650, Tovetumab, Trastuzumab, Trastuzumab emtansine, TRBS07, Tremelimumab, Tucotuzumab celmoleukin, Ublituximab, Urelumab, Vandortuzumab vedotin, Vantictumab, Vanucizumab, Veltuzumab, Volociximab, Vorsetuzumab mafodotin, Votumumab, Zalutumumab, and Zatuximab.

The skilled addressee would understand that this is by no means an exhaustive list, and that other anticancer antibodies and/or chemotherapeutic agents developed in the future may be suitable for use in the methods of the present invention.

In embodiments of the invention, the method comprises the administration of a composition comprising two or more bioactive agents.

In other embodiments of the invention, the bioactive agent is a curcuminoid. By curcuminoid is meant a linear diarylheptanoid, such as curcumin or derivatives thereof which comprise the compound diferuloymethane. The curcuminoid of the present invention may be obtained from commercially available sources and/or prepared, isolated or derived for source material. The curcuminoid may be a synthetic compound, a natural compound or a semi-synthetic compound. For example, the compound may be chemically synthesised, isolated from a plant source, or may be in the form of an extract from an a or plant source, or combinations thereof.

Curcuminoid extracts from plant sources may be prepared by methods known to those skilled in the art and may include processes such as water extractions, chromatographic extractions, solvent extractions, lipid-phase and solid phase extractions, precipitations steps, drying steps, and clarification and purification steps.

The most common curcuminoid is curcumin, which is a natural polyphenol derived most abundantly from turmeric (Curcuma longa). Curcumin has pleiotropic molecular effects and has been used in traditional medicine to treat various disorders, and in particular, inflammation. In one or more embodiments of the present invention, the curcuminoid is selected from the group consisting of curcumin, demethoxycurcumin and bisdemethoxycurcumin. In embodiments of the present invention, the curcuminoid is in the form of an extract from a plant source, wherein the plant source is selected from the group consisting of turmeric, Devil's Claw, White Willow, ginger, grape seed extract, Giant knotweed and green tea.

The NK1R antagonist (or derivative or salt thereof) and the at least one bioactive agent may be combined at any ratio that will enhance the therapeutic efficacy. In embodiments of the present invention, the ratio of NK1R antagonist (or derivative or salt thereof) and bioactive agent is from about 100:1 to about 1:100.

In alternative embodiments, the methods of the invention comprise treating a human subject having a cancer that is refractory to treatment. In the context of cancer (and proliferative diseases generally), ‘refractory’ means ‘resistant’ or ‘intractable’. Thus, a cancer that is refractory upon treatment with at least one bioactive agent (particularly a chemotherapeutic agent) shows no or only weak antiproliferative response (i.e. no or only weak inhibition of cancer growth). As such, the methods of the invention comprise treating a human subject having cancer that is refractory to treatment with at least one bioactive agent other than an NK1R antagonist (or derivative or salt thereof), preferably wherein the NK1R antagonist is aprepitant. That is, the human subject having cancer has previously been treated with at least one bioactive agent other than an NK1R antagonist (or derivative or salt thereof), and the at least one bioactive agent has had no or weak impact on cancer growth.

In other preferred embodiments of the present invention, the additional bioactive agent is a prodrug. A prodrug is a drug derivative of an active drug that may be inert or have lower toxicity than the active drug, which may be processed to the active drug in vivo. The processing may occur by enzymatic of chemical means, and/or the processing may be triggered by biological signals, such as changes in pH, and/or in response to binding a cellular component. A variety of prodrug-like substances are known in the art and an exemplary set of such compounds are disclosed elsewhere herein, where such compounds can include gancyclovir, 5-fluorouracil, 6-methylpurine deoxyriboside, cephalosporin-doxorubicin, 4-[(2-chloroethyl) (2-mesyloxyethyl)amino]benzoyl-L-glutamic acid, acetaminophen, indole-3-acetic acid, CB 1954, 7-ethyl-10-[4-(1-piperidino)-1-piperidino]carbonyloxycamptothecin, bis-(2-chloroethyl)amino-4-hydroxyphenyl-aminomethanone 28, 1-chloromethyl-5-hydroxy-1,2-dihydro-3H-benz[e]indole, epirubicin-glucuronide, 5′-deoxy-5-fluorouridine, cytosine arabinoside, linamarin, and a nucleoside analogue (e.g., fluorouridine, fluorodeoxyuridine, fluorouridine arabinoside, cytosine arabinoside, adenine arabinoside, guanine arabinoside, hypoxanthine arabino side, 6-mercaptopurineriboside, theoguanosine riboside, nebularine, 5-iodouridine, 5-iododeoxyuridine, 5-bromodeoxyuridine, 5-vinyldeoxyuridine, 9-[(2-hydroxy) ethoxy]methylguanine (acyclovir), 9-[(2-hydroxy-1-hydroxymethyl)-ethoxy]methylguanine (DHPG), azauridien, azacytidine, azidothymidine, dideoxyadenosine, dideoxycytidine, dideoxyinosine, dideoxyguanosine, dideoxythymidine, 3′-deoxyadenosine, 3′-deoxycytidine, 3′-deoxyinosine, 3′-deoxyguanosine, 3′-deoxythymidine).

In another aspect of the invention, there is provided the use of a NK1R antagonist (or derivative or salt thereof) in the preparation of a medicament for the treatment or prevention of cancer.

### Compositions and Medicaments, Dosages and Administration

The compositions, compounds, and medicaments of the present invention can be administered by oral, topical or parenteral routes, including intravenous, intramuscular, intraperitoneal, and subcutaneous. They may be delivered by injection directly into a tumour. They may also be administered to organs, tissues and cells ex vivo.

For intramuscular, intraperitoneal, subcutaneous and intravenous use, the compositions and medicaments of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

For oral administration, the compositions of the invention will generally be provided in the form of tablets or capsules or as an aqueous solution or suspension. Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are examples of suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatine capsules in which the active ingredient is mixed with a solid diluent and soft gelatine capsules wherein the active ingredient is mixed with water or an oil such as peanut oil, liquid paraffin or olive oil.

For topical administration, the compositions of the invention will generally be provided in the form of liquid, lotion, emulsion, mousse, paste, cream, ointment, or gel, and may be useful for the treatment of prevention of skin cancers or cancer near the skin surface, such that the NK1R antagonist (or a derivative or pharmaceutically acceptable salt thereof) would be able to diffuse to affected cells. Compositions of the invention for topical administration also include pharmaceutically acceptable preservatives, humectants, emollient, moisturisers, consistency factors, chelating agents, excipients, diluents and colouring agents.

Effective doses of the compositions, compounds and medicaments used in the present invention may be ascertained by conventional methods, and will generally be dependent on the specific therapeutic agent used, and delivery method. The specific dosage level required for any particular subject will depend on a number of factors, including the severity of the condition being treated, the route of administration and the weight of the subject.

For example, 1 unit dose may include about 100 mg to about 10000 mg of aprepitant, or alternative form(s), derivative(s), or salt(s) thereof, or combinations thereof, and/or source(s) thereof described in any aspect and/or example of the invention. In another example, the unit dose includes about 200 mg to about 10000 mg, about 250 mg to about 5000 mg, about 250 mg to about 4000 mg, about 250 mg to about 3000 mg, about 250 mg to about 2500 mg, about 250 mg to about 2000 mg, about 500 mg to about 2500 mg, about 1000 to about 2000 mg, about 500 mg to about 2000 mg, about 1000 mg to about 2000 mg, about 1500 mg to about 2500 mg, about 5 mg to about 1000 mg, about 100 mg to about 1000 mg, about 200 mg to about 2000 mg, about 50 mg to about 2500 mg, about 500 mg to about 1000 mg, about 1000 mg to about 1750 mg, about 250 mg to about 1750 mg, about 500 mg to about 1750 mg, about 250 mg to about 2500 mg, about 500 mg to about 5000 mg, about 1500 mg to about 3000 mg, about 2000 mg to about 3500 mg, about 2500 mg to about 4000 mg, about 3000 mg to 4500 mg, about 3500 mg to about 5000 mg, about 4000 mg to about 5500 mg, about 4500 mg to about 6000 mg, about 5000 mg to 6500 mg, about 5500 mg to 7000 mg, about 6000 mg to 8000 mg, about 6500 to 9000 mg, or about 7000 to about 10000 mg. Preferably, the unit dose includes about 250 mg, about 500 mg, about 750 mg, about 1000 mg, about 1250 mg, about 1500 mg, about 1750 mg, about 2000 mg, about 2250 mg, about 2500 mg, or about 5000 mg of the NK1R antagonist (preferably aprepitant) or alternative form(s), derivative(s), or salt(s) thereof, or combinations thereof, and/or source(s) thereof described in any aspect and/or example of the invention.

The unit does may be expressed as mg/kg, and in embodiments of the invention, this includes a unit does of about 3 mg/kg, about 4 mg/kg, about 5 mg/kg, about 8 mg/kg, about 10 mg/kg, about 15 mg/kg, about 20 mg/kg, about 25 mg/kg, about 30 mg/kg, about 40 mg/kg, or about 50 mg/kg, or about 3 mg/kg to about 30 mg/kg, about 5 mg/kg to about 35 mg/kg, about 10 mg/kg to about 40 mg/kg, about 5 mg/kg to about 15 mg/kg, about 15 mg/kg to about 30 mg/kg, about 30 mg/kg to about 50 mg/kg, about 50 mg/kg to about 70 mg/kg, about 70 mg to about 90 mg/kg, or about 90 mg/kg to about 110 mg/kg of the NK1R antagonist (preferably aprepitant) or alternative form(s), derivative(s), or salt(s) thereof, or combinations thereof, and/or source(s) thereof described in any aspect and/or example of the invention.

The unit dose may be administered once, twice, three, four or five times daily, or may be administered every second or third day, or once every week, once every two weeks or once every four weeks.

In preferred embodiments, the unit dose is administered once, twice or three time daily, for at least 3 days, for 1 week, for 2 weeks, for 3 weeks, for 4 weeks, for 2 months, for 3 months, for 4 months, for 6 months or for up to a year, or for specified periods during the duration of cancer treatment.

Preferably, the NK1R antagonist (preferably aprepitant) or alternative form(s), derivative(s), or salt(s) thereof, or combinations thereof, and/or source(s) thereof, is administered for a period to at least slow, delay or halt the proliferation of cancerous cells, or to at least slow, delay or halt metastasis of a cancer, to slow, delay or halt the increase in a tumour size that would ordinarily occur from the cellular proliferation within the tumour, or at least reduce tumour size, or reverse the progression of one or more tumours, at least temporarily.

In some embodiments, the compositions, compounds and medicaments may be administered to a subject in isolation or in combination with other additional bioactive agent(s). In such embodiments the administration may be simultaneous or sequential.

Typically, in treatment applications, the compositions, compounds and medicaments may be administered for the duration of the cancer. Further, it will be apparent to one of ordinary skill in the art that the optimal quantity and spacing of individual dosages can be determined by the nature and extent of the disease state or condition being treated, the form, route and site of administration, and the nature of the particular subject being treated. In subject where the aprepitant is well tolerate, it may be possible to administer higher doses, more than once daily, for longer periods of time to achieve superior therapeutic results.

Some embodiments of the present invention may involve administration of the composition or medicament in multiple, separate doses. Accordingly, the methods of treatment and prevention described herein encompass the administration of multiple separated doses to a subject, for example, over a defined period of time.

The compositions and medicaments of this invention may also be useful in combination (administered together or sequentially) with one or more additional therapeutic treatments such as radiation therapy, and/or one or more additional therapeutic agents selected from the group consisting of different types of chemotherapy drugs, anti-tumour antibiotics, topoisomerase inhibitors, mitotic inhibitors, corticosteroids, targeted therapies, differentiating agents, hormone therapy and immunotherapy.

Further examples of the invention are described below. However, it should be noted that the invention should not be limited to these examples, and that the invention is susceptible to variations, modifications and/or additions other than those specifically described, and it is to be understood that the invention includes all such variations, modifications and/or additions which fall within the scope of the claims.

### EXAMPLES

### Example 1—In Vitro Studies

For in vitro studies, Aprepitant (CAS No 170729-80-3, Sigma-Aldrich), fosaprepitant (B1272, ApexBio), fosaprepitant dimeglumine salt (CAS No 265121-04-8, Selleck Chemicals), Netupitatnt (CAS No 290297-26-6, Sigma-Aldrich), Imatinib mesylate (CAS No 220127-57-1, Selleck Chemicals), Vemurafenib (CAS No 918504-65-1, Selleck Chemicals), Venetoclax (CAS No 1257044-40-8, Selleck Chemicals) and Palbociclib (PZ0383, Sigma-Aldrich) were used. All were administrated at different doses in a variety of cell lines as shown in Table 1.

TABLE 1
In vitro cell lines

| | | | Cell |
|---|---|---|---|
| Cell line | Specie | Disease | culture media |
| 3LL | Mouse | Lewis Pulmonar Carcinoma | DMEM |
| B16F10 | Mouse | Melanoma | DMEM |
| MATBIII | Rat | Mammary gland | McCoy's 5A |
| | | adenocarcinoma | |
| CT26WT | Mouse | Colon carcinoma | RPMI-1640 |
| RT112 | Human | Urinary bladder | RPMI-1640 |
| | | carcinoma | |
| 4T1 | Mouse | Mammary gland | RPMI-1640 |
| | | adenocarcinoma | |
| Renca | Mouse | Renal adenocarcinoma | RPMI-1640 |
| PTEN-P8 | Mouse | Prostate cancer tumor | DMEM |
| L1210 | Mouse | Lymphocytic leukemia | DMEM |
| Jurkat | Human | Acute T cell leukemia | RPMI-1640 |
| EL4 | Mouse | Lymphoma | DMEM |
| A375 | Human | Melanoma | DMEM |
| A549 | Human | Lung carcinoma | F-12K |
| HT-29 | Human | Colorectal adenocarcinoma | McCoy's 5A |
| BxPc3 | Human | Pancreas adenocarcinoma | RPMI-1640 |
| T47D | Human | Mammary gland- ductal | RPMI-1640 |
| | | carcinoma | |
| A20 | Mouse | B cell lymphoma | RPMI-1640 |
| MCF7 | Human | Breast adenocarcinoma | DMEM |
| SKBr3 | Human | Breast adenocarcinoma | McCoy's 5A |
| MDA-MB-231 | Human | Breast adenocarcinoma | Leibovitz's |
| Malme-3 | Human | Skin fibroblast | McCoy's 5A |

Cell lines used for viability assays in vitro. Species, type of tumour and the medium used for cell culture of each cell line are shown. Cell culture media used are Dulbecco's Modified Eagle Medium (DMEM), McCoy's 5A Modified Medium (McCoy's 5A), RPMI Medium 1640 (RPMI-1640), Kaighn's Modification of Ham's F-12 Medium (F-12K) and Leibovitz's Medium (Leibovitz's).

21 Cell lines of three different species (mouse, rat and human) were used in this study (Table 1). All of them were maintained in culture in their respective culture media (Gibco, Thermo Fisher Scientific) supplemented with 10% fetal bovine serum (FBS) (Gibco, Thermo Fisher Scientific), 2 mM L-Glutamine (Sigma-Aldrich, Merk), and Peni-Strep (50 U/ml and 50 μl/ml; Gibco, Thermo Fisher Scientific) in a humidified incubator at 37° C. and 5% CO2. Malme-3 cell line culture media was supplemented with 15% FBS and T47D cell line culture media was supplemented with human insulin 10 μg/μl (19278 Sigma Aldrich, merk).

The cytotoxicity of the drugs in culture was evaluated using the 3-(4,5-Dimethyl-2-thiazolyl)-2,5-diphenyl-2H-tetrazolium bromide (MTT) reduction assay. The assay assesses cell viability through the indirect measurement of the activity of reductases that convert yellow soluble MTT into purple insoluble formazan salts. As MTT can only be reduced when these enzymes are active, the reaction is used as an indicator of the cell metabolic competence and, therefore, of viability.

A variable number of cells (depending on the cell line) were seeded in 96 multi-well plates and grown during 24 Hours Cell Viability. The following day the culture medium was aspirated and fresh culture medium with the drug dissolved was added to the wells. After 24 Hours Cell Viability or 48 Hours Cell Viability cell viability was measurement by adding 10 μl of a 5 mg/ml MTT solution (M5655; Sigma-Aldrich) and 100 μl of a Hcl 0.01M-SDS 10% solution 4 hours later. After 24 Hours Cell Viability, the absorbance was measured at 595 nm, using a multi-well plate reader (iMark, Bio-rad).

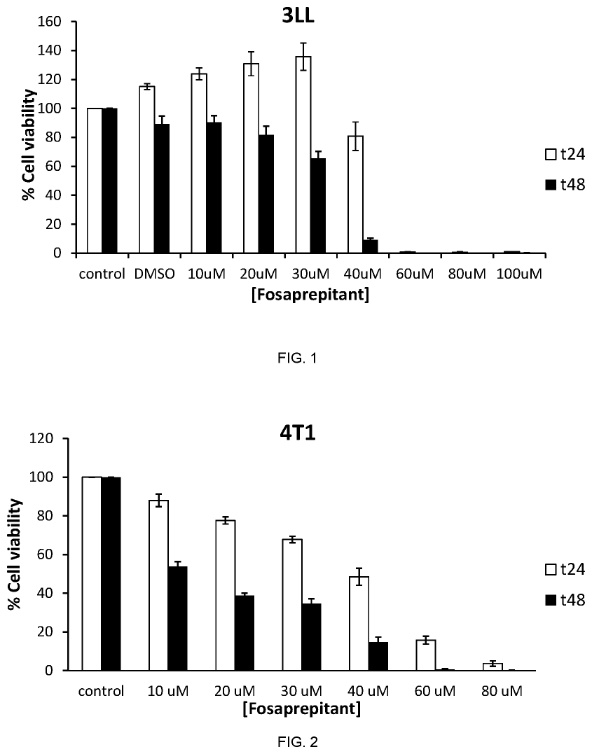

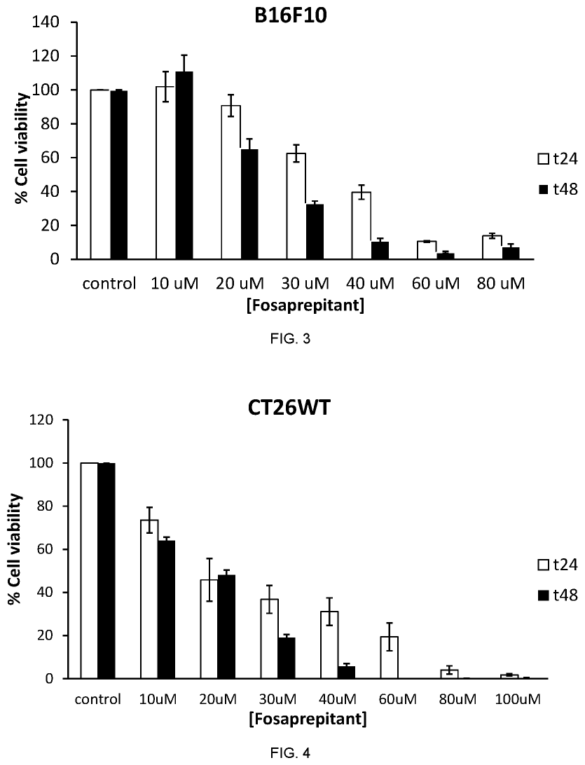

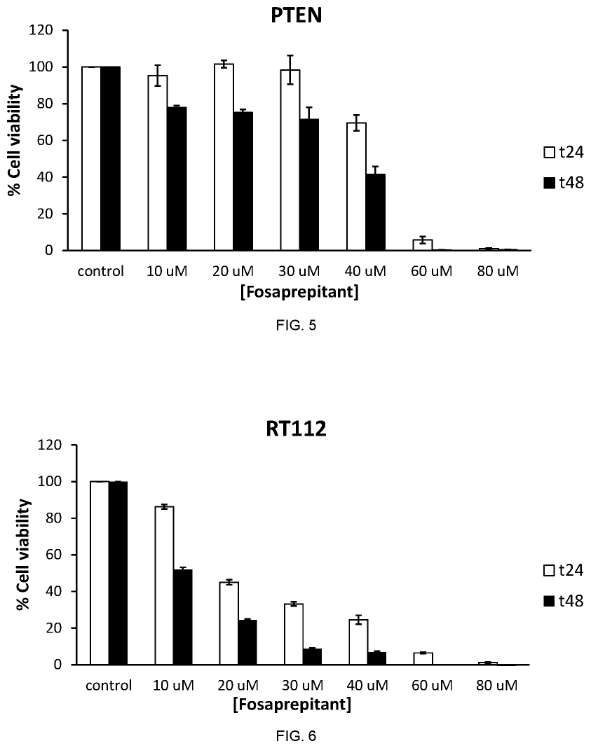

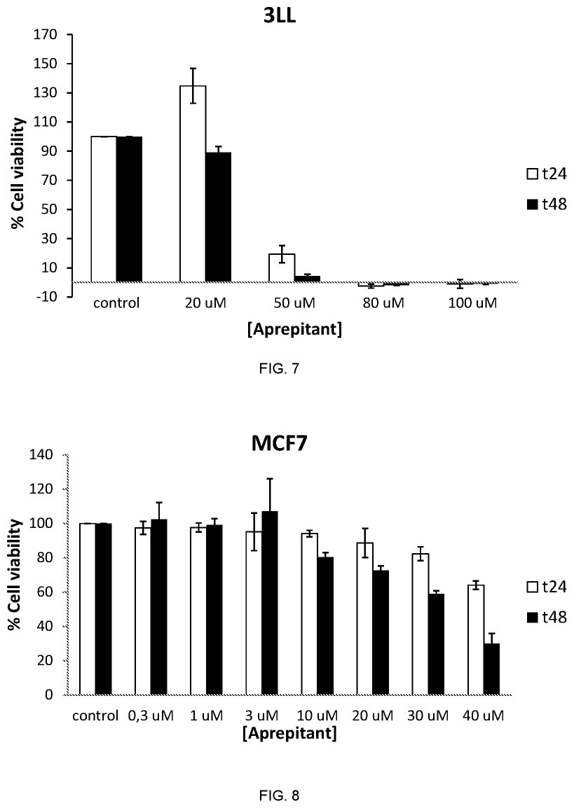

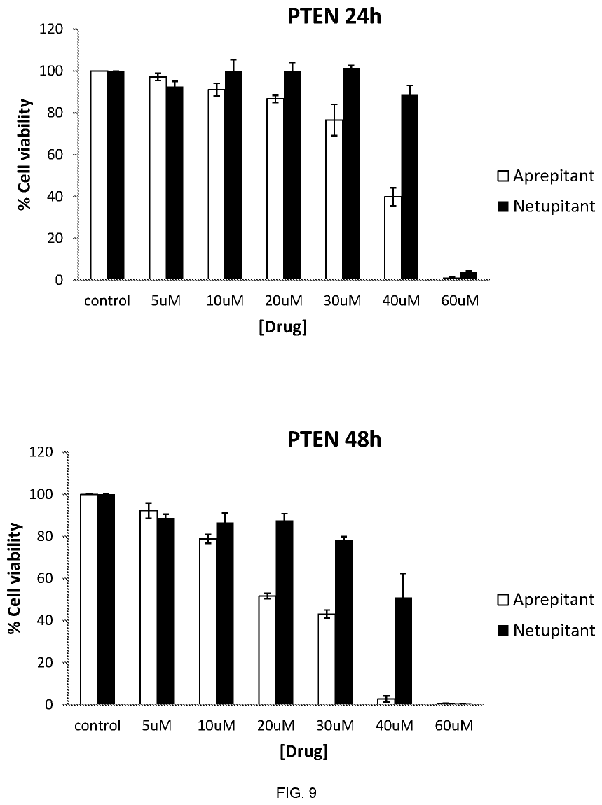

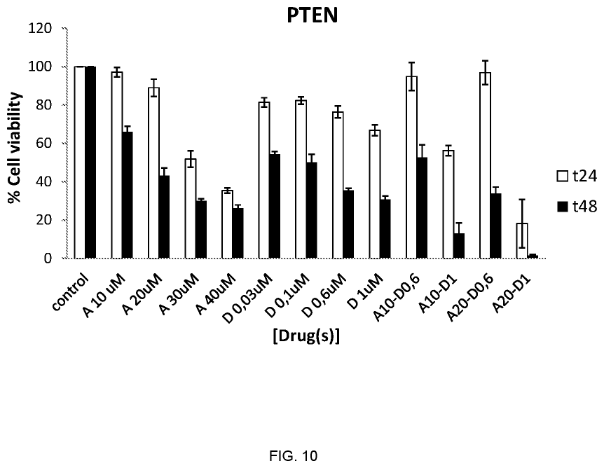

The results of several cytotoxicity studies is shown in FIGS. 1-9. Specifically, the results show the ability of NK1R antagonists, particularly fosaprepitant, aprepitant and netupitant, exhibited anti-cancer effects in vitro. FIG. 10 shows that the combination of aprepitant and doxorrubicine surprisingly acted synergistically against mouse prostate cancer tumour cells.

By contrast, no significant effect of the NK1R antagonists was observed on non-tumour human fibroblast cells.

### Example 2—In Vivo Studies

For in vivo studies, the drugs fosaprepitant dimeglumine salt (CAS No 265121-04-8, Selleck Chemicals), Cerenia® (Maropitant citrate injectable solution; Zoetis US), InVivoMAb Anti-mouse CTLA-4 (CD152) (BE0032, Bio cell), InVivoMAb Anti-mouse PD-1 (CD279) (BE0146, Bio cell) and Rat IgG2a k isotype (ref BE0089) were used.

B16F10 cells (1×10⁶ per mouse) were subcutaneously injected into the right flank of C57BL/6J female mice (Charles River) with matrigel (Matrigel Membrane Matrix, Corning) at 50% v/v. Tumour growth was evaluated every 2/3 days. Animal vital parameters were monitored daily. When the tumours reached an average volume of 100-150 mm³, mice were randomised into cohorts of 6 animals and treatments were administered for 7-9 days. Groups and regimens for each experiment are described in Table 2. Animals of the control group were dosed with equal volume of vehicle. Animals were euthanized according to institutional guidelines and tumour samples were excised. Tumours were snap-frozen in OCT medium (Sakura Tissue Tek) or fixed in PFA and embedded in paraffin. Several internal organs were also excised and fixed in PFA for future studies.

A total of 217 autopsies were performed on mice of C57BL/6J female mice (provided by the Charles River company). They were treated with immune control point inhibitors such as anti-CTLA-4 antibodies and anti-PD1 antibodies, Isotype antibodies as control of anti-CTLA-4, Isotype antibodies as control of anti-CTLA-4, Isotype antibodies as control of anti-PD1 and NK1 receptor antagonists. Cases were also included as a control to which no treatment was applied.

During the autopsy of each animal, samples were taken from the different organs: Central Nervous System, Thymus, Heart, Lungs, Liver, Spleen, Kidneys, Adrenal Glands, Stomach, Large Intestine, Small Intestine and genital apparatus.

The following table shows the cases of autopsy and treatment applied to each one:

TABLE 2
Evaluation of NK1R antagonists in mouse xenograft model

| Drug (s) Dosage | N | % |
|---|---|---|
| Anti CTLA-4 10 mg/kg | 4 | 1.8 |
| Anti CTLA-4 3 mg/kg | 7 | 3.2 |
| Anti CTLA-4 5 mg/kg | 13 | 6.0 |
| Anti CTLA-4 8 mg/kg | 4 | 1.8 |
| Anti PD-1 10 mg/kg | 4 | 1.8 |
| Anti PD-1 8 mg/k | 6 | 2.8 |
| Anti PD-1 8 mg/kg | 6 | 2.8 |
| Control | 25 | 11.5 |
| Fosaprepitant 30 mg/kg | 22 | 10.1 |
| Fosaprepitant 30 mg/kg + Isotype 5mg/kg | 6 | 2.8 |
| Fosaprepitant 30 mg/kg + Anti CTLA-4 5mg/kg | 6 | 2.8 |
| Fosaprepitant 30 mg/kg + Anti PD1 8 mg/kg | 12 | 5.5 |
| Fosaprepitant 30 mg/kg + Isotype 8 mg/kg | 12 | 5.5 |
| Isotype 5 mg/kg | 6 | 2.8 |
| Isotype 8 mg/kg | 12 | 5.5 |
| Maropitant 30 mg/kg | 12 | 5.5 |
| Maropitant 30 mg/kg + Anti CTLA-4 5 mg/kg | 6 | 2.8 |
| Maropitant 30 mg/kg + Anti PD1 mg/kg | 6 | 2.8 |
| Maropitant 30 mg/kg + Isotype 5 mg/kg | 6 | 2.8 |
| Maropitant 30 mg/kg + Isotype 8 mg/kg | 6 | 2.8 |
| Netupitant 10 mg/kg | 7 | 3.2 |
| Netupitant 30 mg/kg | 13 | 6.0 |
| Netupitant 30 mg/kg + Anti PD1 8 mg/kg | 6 | 2.8 |
| Netupitant 30 mg/kg + Isotype 8 mg/kg | 6 | 2.8 |
| Netupitant 60 mg/kg | 4 | 1.8 |
| Total | 217 | 100.0 |

The samples were fixed in 4% formalin, processed in the Leica TP1020 tissue processor and embedded in paraffin blocks. Subsequently, histological sections were made that were extended on slide and stained with hematoxylin-eosin staining for microscopic evaluation.

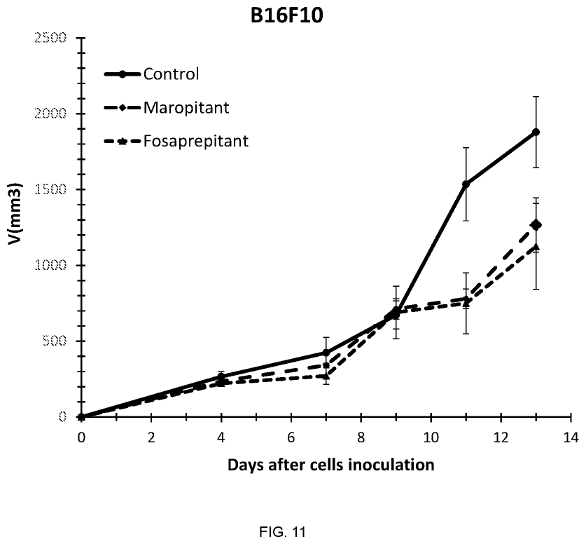

The results of FIG. 11 exemplify a general trend with NKR1 antagonists. Specifically, treatment with drugs fosaprepitant-dimeglumine or Maropitant reduced tumoral growth in mouse melanoma model.

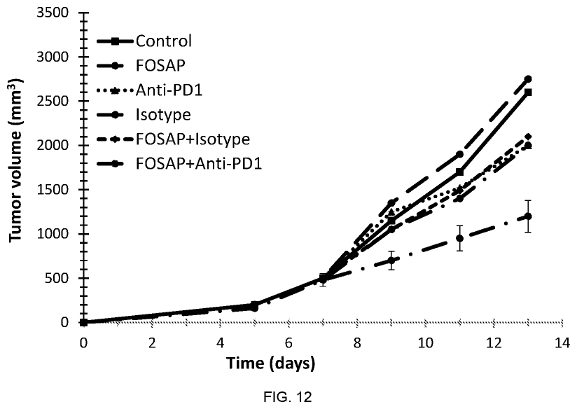

FIG. 12 shows that the combination of fosaprepitant-dimeglumine and anti-PD1 therapies shows enhanced antitumoral activity compared with single treatments in mouse melanoma model.

### Example 3—Compassionate Use Protocol

A patient (Male, date of birth 30 Dec. 1969) was diagnosed with osteoclastic osteosarcoma clinical stage IV, after histopathological study of a lesion in the proximal third of the left radius. A pathology analysis defined the cancer as a “High grade sarcoma”, together with the determination by immunohistochemistry of the markers: Vimentin, FLI1, EMA and ERG (Positive) and S-100, CD34, CD31, CK-AE1AE3 Negative.

Surgical resection of the primary lesion was performed in August 2021 and first line treatment was initiated in October 2021 with cisplatin and doxorubicin after finding local recurrence with areas of chondroid differentiation of the left radius as well as metastatic lesions in both lungs. After completing 6 cycles he showed a partial response with reduction of the size and number of lung lesions and complete metabolic response of the residual lesion in the forearm.

After a multidisciplinary assessment, surgical resection of the residual lesion was discouraged and SBRT radiotherapy was indicated instead for the residual lesion in the left arm (45.5 Gy; Mar. 9-Apr. 12, 2022) as well as for the pulmonary metastatic lesions in the right lower lobe (50 Gy; Mar. 16-22, 2022).

In April 2022, progression was demonstrated by contrasted CT scan at the expense of lung lesion growth; the second line of treatment with ifosfamide and etoposide was initiated with good tolerance to treatment and achieved a partial response with evidence of a decrease in the size of the lesions without other hypermetabolic foci suggestive of malignancy.

On Jul. 18, 2022 resection of the right lung lesions was performed. The anatomopathological study reported: 6 nodules with reparative fibrous changes, without evidence of residual tumour, one lymph node without evidence of tumour infiltration, 3 nodules infiltrated by high-grade sarcoma with post-treatment changes (fibrosis) and one intercisural lesion with marked fibrohistocytic reaction and foci of hemorrhage and infiltration by high-grade sarcoma.

Postoperatively, the patient required placement of PleurX pleural drainage, due to persistent right pneumothorax, for which chemical pleurodesis (thalcage) was indicated using the Slurry technique through the drainage. Pleural fluid cytology showed no atypical cells.

A next-generation genomic sequencing study (Foundation OneHEME) was indicated and reported: microsatellite stability, TMB: 2 muts/Mb, rearrangement of intron 4 of NF1, equivocal amplification of CDK-6, FANCA Q1437*, without identifying therapies with clinical relevance for this neoplasm, selumetinib and trametinib as clinically relevant treatments in another type of cancer due to possible sensitivity to MEK inhibitors in patients with NF1 inactivation based on isolated clinical data and aprepitant as an experimental drug without scientific evidence in humans only in cell lines and murine models.

The postoperative re-evaluation thoracoabdominal CT scan on Aug. 26, 2022 showed progression at the expense of pulmonary and pleural lesions, so a third line of chemotherapy with gemcitabine and docetaxel was started. The patient had poor tolerance to the scheme so he was offered repeat treatment with ifosfamide and etoposide, with a bleak prognosis for life and function.

On Dec. 8, 2022 a PET/CT was performed which reported abnormal glycolytic metabolism in multiple right pleural implants and bilateral pulmonary nodules, right pulmonary collapse in relation to tumour activity. The left lung presented with solid nodules, the largest with spiculated edges measuring 16.7 mm in the posterior apical segment and one of 5.4 mm in the upper segment of the lower lobe. In the right lung there was a solid nodule with regular borders measuring 15 mm parahilar in the middle lobe. There was no evidence of tumour activity in the left forearm. Clinically the patient reported hypersomnia, nausea, dizziness and constipation, ECOG 1 was determined and a global EORT CQL-C30 score of 50%.

On Dec. 9, 2022 the patient was approved for compassionate use of aprepitant at a dose of 4200 mg orally divided into 3 doses (the total daily dose being 4200 mg) along with ifosfamide and etoposide as a fourth line of treatment. Close clinical surveillance of the patient was initiated to monitor quality of life, functional status and manifestation of adverse reactions to treatment, as well as periodic serological surveillance and measurement of response to treatment by imaging studies using the RECIST 1.1 criteria.

On Jan. 9, 2023 a follow up PET/CT was performed. In the right pleura there was a solid nodule with irregular borders of 42 mm in diameter to hilar in the middle lobe. In the left lung there were nodules in the posterior apical segment of 21 mm, in the left upper lower lobe one of 16 mm, two basal, the largest of 20 mm. Liver, gallbladder, pancreas, spleen, adrenal glands and kidneys of normal characteristics, without focal lesions or abnormal contrast enhancement. No mesenteric or retroperitoneal adenopathy was detected.

On Feb. 6, 2023 there is a slight increase in the size of bilateral pulmonary nodules and masses of right predominance. Left upper nodule: 21 mm, pulmonary nodule in left lower nodule 10 mm, pulmonary nodule in left lower lobe 16.5 mm. No pleural or pericardial effusion was detected, and the liver was free of focal lesions, pleural implant lesions imprinting on the hepatic capsule, without adenopathies in abdominopelvic lymph node chains.

### Analysis

The above describes a 53-year-old male diagnosed with osteoclastic osteosarcoma in clinical stage IV, with current management based on ifosfamide, etoposide and compassionate use of aprepitant. By Feb. 15, 2023 the patient had maintained a stable disease type response according to RECIST 1.1 criteria as well as clinical and serological surveillance.

The patient has preserved his functional status by retaining an ECOG of 1 since the start of follow-up along with a Karnofsky of 70%. His quality of life has also been preserved, as measured by the EORTC QLQ-C30 questionnaire, obtaining an overall score of 50% since the start of therapy. Likewise, the patient's symptomatology has remained constant with no increase in symptoms when evaluated by the Edmonton scale, with adequate tolerance thanks to the supportive treatment.

When comparing the patient's evolution with the expected course of the disease, which is characterised by a rapid progression and a low survival rate, it can be concluded that the use of aprepitant has had a positive impact on the patient. Aprepitant has managed to maintain in all aspects (clinical, serological and imaging) a stable disease-like response.

### Example 4—Trial to Determine Maximum Tolerable Dose (MTD) and Dose Limiting Toxicities (DLT) of Multiple Ascending Doses of Aprepitant

The study intends to enrol participants who have advanced solid tumours and have failed to respond effectively to first-line therapy. Further, this study intends to extend the current knowledge relating to aprepitant and its use chronically as a possible mode of treatment for solid tumours. During the dose escalation stage, aprepitant will be dosed initially as a single oral dose for Cohort 1, increasing to twice a day to allow for dosing of up to 1000 mg per day, and then three times a day for later cohorts where doses will increase up to 5000 mg per day. The increase in dose will be based on a critical examination of safety data following 4-weeks administration, to identify the occurrence of dose limiting toxicities. The starting dose proposed is based upon existing clinical and non-clinical experience, and will represent a doubling of the first dose when used for preventative treatment of cancer induced nausea and vomiting. Dosing will be increased until the MTD has been determined.

Whereas the use of aprepitant for the preventative treatment of nausea and vomiting relies on a limited low dose (once daily dose for 3 days), this study will also characterise the pharmacokinetics of the higher doses of aprepitant, not only following a single dose, but also steady state kinetics. Clinically, it is anticipated that aprepitant will be administered at a dosage ranging between about 3,000 mg per day to about 5,000 mg per day, where the treatment duration is expected to average between 4 to 8 months.

1. A method of treating or preventing cancer in a human subject, the method comprising the step of administering to said human subject a composition comprising a therapeutically effective amount of an NK1R antagonist, or a derivative or pharmaceutically acceptable salt thereof.

2. The method according to claim 1, wherein the NK1R antagonist is selected from aprepitant, rolapitant, casopitant, fosaprepitant, netupitant, maropitant, or a derivative or pharmaceutically acceptable salt thereof.

3. The method according to claim 1, wherein the NK1R antagonist or a derivative or pharmaceutically acceptable salt thereof is administered at a dose of about 100 mg to about 10000 mg per day.

4. The method according to claim 1, wherein the NK1R antagonist or a derivative or pharmaceutically acceptable salt thereof is administered at a dose of about 1500 mg to 6000 mg per day.

5. The method according to claim 1, wherein the NK1R antagonist or a derivative or pharmaceutically acceptable salt thereof is administered at a dose of about 3000 mg to about 5000 mg per day.

6. The method according to claim 1, wherein the NK1R antagonist or a derivative or pharmaceutically acceptable salt thereof, is administered for a period to at least slow, delay or halt the proliferation of cancerous cells, to at least slow, delay or halt metastasis of a cancer, to at least slow, delay or halt the increase in a tumour size that would ordinarily occur from the cellular proliferation within the tumour, or at least reduce tumour size, or reverse the progression of one or more tumours, at least temporarily.

7. The method according to claim 1, wherein the cancer is solid tumour or a blood cancer.

8. The method of claim 7, wherein the administration of the NK1R antagonist, or a derivative or pharmaceutically acceptable salt thereof:
induces cancer cell apoptosis,
induces cancer cell death,
reduces tumoral growth, and/or
reduces tumour size.

9. The method according to claim 1, wherein prior to the administration of the NK1R antagonist, or a derivative or pharmaceutically acceptable salt thereof, the cancer has been refractory to treatment with at least one cancer treatment.

10. The method according to claim 1, wherein the method further comprises administering the NK1R antagonist, or a derivative or pharmaceutically acceptable salt thereof, in combination with at least one bioactive agent.

11. The method of claim 10, wherein the at least one bioactive agent is an anticancer drug, chemotherapeutic agent, curcuminoid, or a prodrug, or a combination thereof.

12. The method of claim 10, wherein the at least one bioactive agent is selected from the group consisting of abiraterone acetate, albumin-bound (nab) paclitaxel, alemtuzumab, altretamine, asparaginase, bendamustine, bevacizumab, bleomycin, bortezomib, brentuximab vedotin, busulfan, cabazitaxel, capecitabine, carboplatin, carmustine, cetuximab, chlorambucil, cisplatin, cladribine, crizotinib, cyclophosphamide, cytarabine (Ara-C), dacarbazine, dactinomycin, dasatinib, daunorubicin, DaunoXome (liposomal daunorubicin), DepoCyt (liposomal cytarabine), docetaxel, Doxil (liposomal doxorubicin), doxorubicin, eribulin mesylate, erlotinib, estramustine, etoposide, everolimus, floxuridine, fludarabine, fluorouracil, gefitinib, gemcitabine, Gliadel wafers, hydroxyurea, ibritumomab, idarubicin, ifosfamide, imatinib, ipilimumab, irinotecan, ixabepilone, lapatinib, lenallidomide, lomustine, mechlorethamine, melphalan, mercaptopurine, methotrexate, mitomycin, mioxantrone, MG132, nilotinib, oxaliplatin, paclitaxel, panitumumab, pazopanib, peginterferon alfa-2b, pemetrexed, pentostatin, pralatrexate, procarbazine, rituximab, romidepsin, ruxolitinib, sipuleucel-T, sorafenib, streptozocin, sunitinib, temozolomide, temsirolimus, teniposide, thalidomide, thioguanine, thiotepa, topotecan, tositumomab, trastuzumab, valrubicin, vandetanib, vemurafenib, vinblastine, vincristine, and vinorelbine.

13. The method according to claim 10, wherein the NK1R antagonist, or a derivative or pharmaceutically acceptable salt thereof, and the at least one bioactive agent act synergistically to treat or prevent said cancer.

14. The method of claim 1, wherein the NK1R antagonist, or a derivative or pharmaceutically acceptable salt thereof, is administered orally or systemically.

15-32. (canceled)

33. The method according to claim 2, wherein the NK1R antagonist is aprepitant, maropitant or fosaprepitant, or a derivative or pharmaceutically acceptable salt thereof.

34. The method according to claim 7, wherein the cancer is selected from sarcoma, carcinoma, lymphoma, leukemia, myeloma, and circulating tumour cells (CTCs).

35. The method according to claim 6, wherein the cancer is selected from the group consisting of: lung cancer; breast cancer; mammary gland adenocarcinoma; melanoma; colorectal cancer; colon carcinoma; prostate cancer; bladder cancer; urinary bladder carcinoma; pancreatic cancer and leukemia.

36. The method according to claim 8, wherein administration of the NK1R antagonist or a derivative or pharmaceutically acceptable salt thereof, reduces tumoral growth, as determined by average tumour volume, by at least about 25%.

37. The method according to claim 36, wherein administration of the NK1R antagonist or a derivative or pharmaceutically acceptable salt thereof, reduces tumoral growth, as determined by average tumour volume, by about 25% to about 65%.

38. The method according to claim 37, wherein administration of the NK1R antagonist or a derivative or pharmaceutically acceptable salt thereof, reduces tumoral growth, as determined by average tumour volume, by at least about 25%, at least about 30%, at least about 40%, at least about 45%, at least about 50%, at least about 55%, at least about 60% or at least about 65%.

39. The method according to claim 38, wherein administration of the NK1R antagonist or a derivative or pharmaceutically acceptable salt thereof, reduces tumoral growth, as determined by average tumour volume, by at least about 26%, at least about 32%, at least about 42%, or at least about 56%.

40. The method according to claim 9, wherein the at least one bioactive agent is an anti-PD1 antibody or a DNA methyltransferase inhibitor.

41. The method according to claim 1, wherein the NK1R antagonist or a derivative or pharmaceutically acceptable salt thereof is administered at a dose of about 500 mg to 5000 mg per day.
